(19) United States
(12) Patent Application Publication
(10) Pub. No.: US 2024/0206748 A1
(43) Pub. Date: Jun. 27, 2024

(54) BLOOD PRESSURE MEASUREMENT DEVICE

(71) Applicant: OMRON HEALTHCARE CO., LTD., Muko-shi (JP)

(72) Inventors: Yoshihide TOKKO, Kyoto (JP); Kosuke ABE, Kyoto (JP); Takashi ONO, Kyoto (JP)

(73) Assignee: OMRON HEALTHCARE CO., LTD., Muko-shi (JP)

(21) Appl. No.: 18/598,010

(22) Filed: Mar. 7, 2024

Related U.S. Application Data
(63) Continuation of application No. PCT/JP23/09679, filed on Mar. 13, 2023.

(30) Foreign Application Priority Data
Aug. 1, 2022 (JP) 2022-122640

(51) Int. Cl. A61B 5/022; A61B 5/00

(52) U.S. Cl. CPC A61B 5/02225; A61B 5/02233; A61B 5/681; A61B 2560/0462; A61B 2562/0247

(57) ABSTRACT

A blood pressure measurement device includes a housing, a flow path plate unit accommodated in the housing and at least partially formed of a conductive material, a pump connected to the flow path plate unit, a pressure sensor connected to the flow path plate unit, a cuff connected to the flow path plate unit and fluidly connected to the pump and the pressure sensor via the flow path plate unit, a printed circuit board accommodated in the housing and including GND, and at least one connection member that connects the GND of the printed circuit board and a part of the flow path plate unit formed of a conductive material.

### CROSS-REFERENCE TO RELATED APPLICATION

This application is a Continuation Application of PCT Application No. PCT/JP2023/009679, filed Mar. 13, 2023 and based upon and claiming the benefit of priority from Japanese Patent Application No. 2022-122640, filed Aug. 1, 2022, the entire contents of all of which are incorporated herein by reference.

### FIELD

The present invention relates to a blood pressure measurement device.

### BACKGROUND

In recent years, blood pressure measurement devices used for measuring blood pressure are being used as means to check health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff by a pressure sensor.

Such a blood pressure measurement device requires a measure against static electricity because electronic components are used. As a measure against static electricity used in existing watches and wearable devices, for example, a technique of connecting a metal housing to GND disclosed in Jpn. Pat. Appln. KOKAI Publication No. 2010 -286260 and a technique of providing a metal member inside and connecting the metal member to GND disclosed in Jpn. Pat. Appln. KOKAI Publication No. 2014 -165046 are known.

### SUMMARY

In the technique of connecting the metal housing to GND explained above, there is a problem in that the housing cannot be used for GND when the housing is made of resin or when the housing is used as an electrode. In the technique of providing the metal member inside and connecting the metal member to GND, an additional member is required for measures against static electricity, causing problems in terms of size and cost.

Therefore, an object of the present invention is to provide a blood pressure measurement device capable of suppressing an increase in size and cost and taking measures against static electricity.

According to one aspect, there is provided a blood pressure measurement device including: a housing; a flow path plate unit accommodated in the housing and at least partially formed of a conductive material; a pump connected to the flow path plate unit; a pressure sensor connected to the flow path plate unit; a cuff connected to the flow path plate unit and fluidly connected to the pump and the pressure sensor via the flow path plate unit; a printed circuit board accommodated in the housing and including GND; and at least one connection member that connects the GND of the printed circuit board and a part of the flow path plate unit formed of the conductive material.

According to this aspect, in the blood pressure measurement device, the GND of the printed circuit board and the flow path plate unit are connected by the connection member. Therefore, it is possible to take measures against static electricity using the flow path plate unit. In the blood pressure measurement device, the flow path plate unit, which is a component necessary for exerting the function of the blood pressure measurement device, is used for measures against static electricity. Therefore, it is possible to prevent the number of components from increasing for the measures against static electricity and prevent an increase in the size and cost of the blood pressure measurement device.

There is provided the blood pressure measurement device in the aspect described above, wherein the flow path plate unit includes: a first flow path plate formed of a metallic material; a second flow path plate formed of a metallic material; and an attachment member that attaches the first flow path plate and the second flow path plate, and the connection member is connected to at least one of the first flow path plate and the second flow path plate.

According to this aspect, the connection member only has to be connected to at least one of the first flow path plate and the second flow path plate formed of the metallic material. There is high flexibility in designing a connection position and the like.

There is provided the blood pressure measurement device in the aspect described above, wherein the attachment member includes a conductive material.

According to this aspect, since the first flow path plate and the second flow path plate are attached by the attachment member containing the conductive material, the first flow path plate, the second flow path plate, and the attachment member are electrically connected, and the volume of the flow path plate unit connected to GND is increased. Since the first flow path plate and the second flow path plate are conducted by the attachment member, the connection member only has to be connected to one of the first flow path plate and the second flow path plate.

According to the present invention, it is possible to provide a blood pressure measurement device capable of suppressing an increase in size and cost and taking measures against static electricity.

### BRIEF DESCRIPTION OF THE DRAWINGS

FIG. 1 is a perspective view illustrating a configuration of a blood pressure measurement device according to a first embodiment of the present invention.

FIG. 2 is a block diagram illustrating the configuration of the blood pressure measurement device.

FIG. 3 is a perspective view schematically illustrating a configuration in a housing of the blood pressure measurement device.

FIG. 4 is an exploded perspective view illustrating configurations of a pump, an on-off valve, a pressure sensor, and a flow path plate unit of the blood pressure measurement device.

FIG. 5 is a cross-sectional view illustrating configurations of the pump, the pressure sensor, the flow path plate unit, a pressing cuff, and a sensing cuff of the blood pressure measurement device.

### DETAILED DESCRIPTION

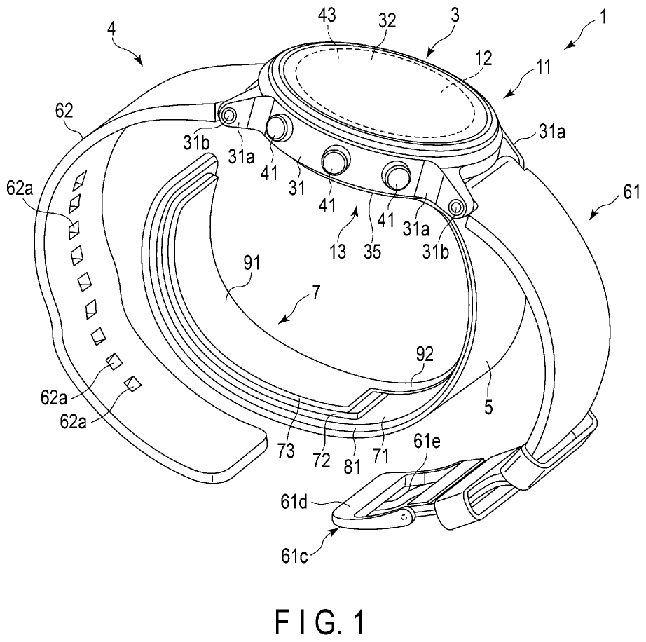

An example of a blood pressure measurement device 1 according to a first embodiment of the present invention is explained below with reference to FIGS. 1 to 5.

FIG. 1 is a perspective view illustrating a configuration of the blood pressure measurement device 1.

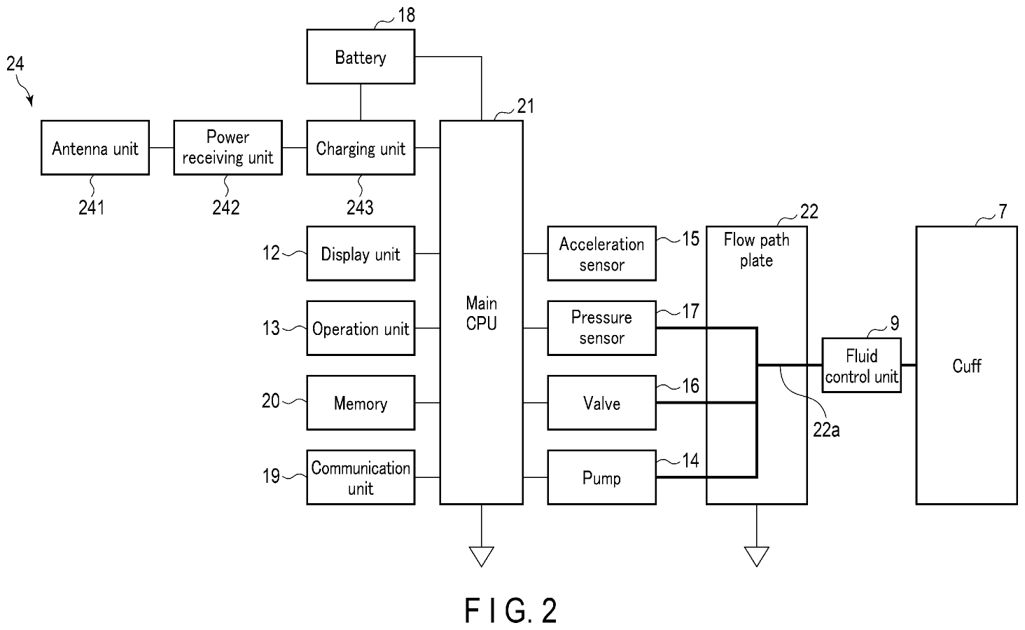

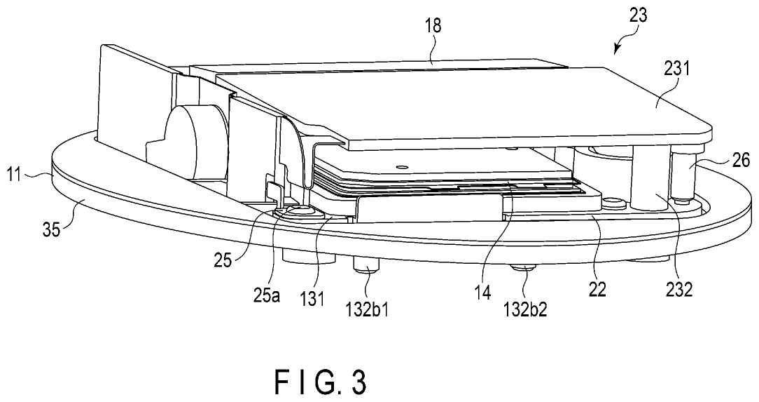

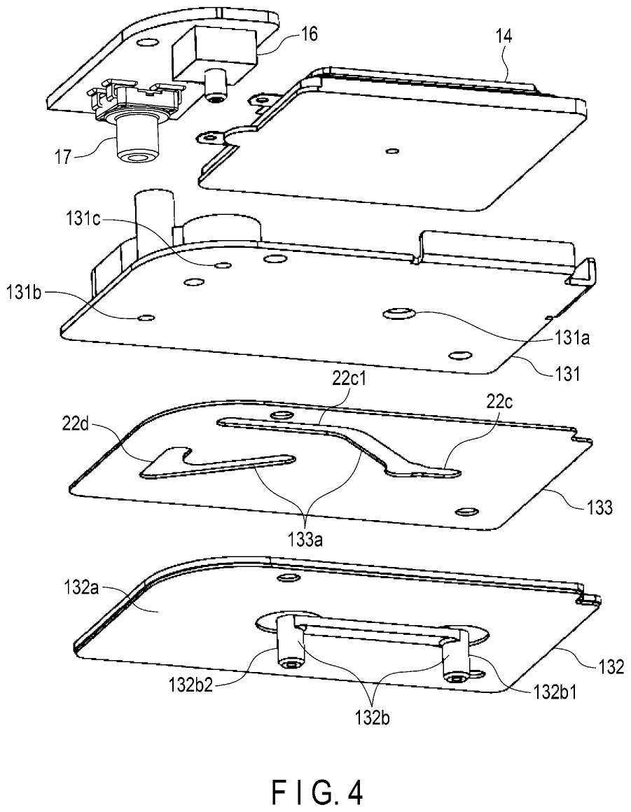

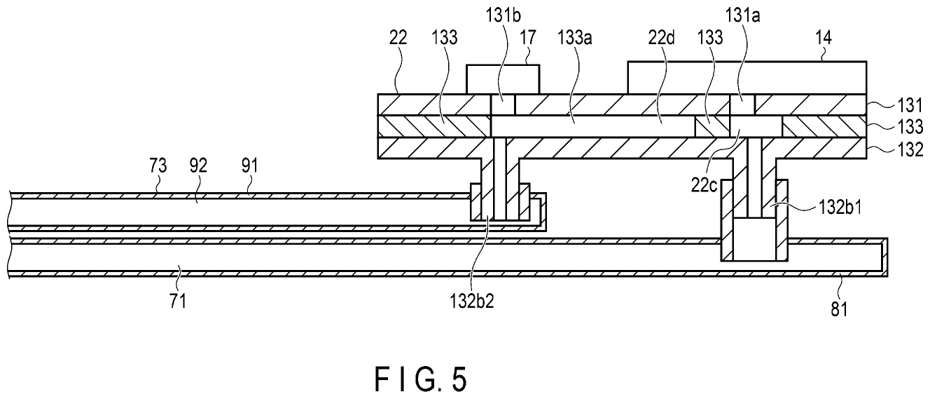

FIG. 2 is a block diagram illustrating the configuration of the blood pressure measurement device 1. FIG. 3 is a perspective view schematically illustrating a configuration in a housing 11 of the blood pressure measurement device 1. FIG. 4 is an exploded perspective view illustrating configurations of a pump 14, an on-off valve 16, a pressure sensor 17, and ta flow path plate unit 22 of the blood pressure measurement device 1. FIG. 5 is a cross-sectional view illustrating configurations of the pump 14, the pressure sensor 17, and the flow path plate unit 22 of the blood pressure measurement device 1.

The blood pressure measurement device 1 is an electronic blood pressure measurement device attached to a living body. In an example of the present embodiment, the blood pressure measurement device 1 is a wearable device worn on a wrist. The blood pressure measurement device 1 is, for example, an electronic blood pressure measurement device having an aspect of measuring blood pressure from an artery.

As illustrated in FIGS. 1 to 5, the blood pressure measurement device 1 includes, for example, a device main body 3, a belt 4, a curler 5, a cuff structure (cuff) 7, and a fluid control unit 9.

The device main body 3 includes, for example, the housing 11, a display unit 12, an operation unit 13, a pump 14, an acceleration sensor 15, an on-off valve 16, a pressure sensor 17, a battery 18, a communication unit 19, a memory 20, a processor 21, a flow path plate unit 22, a mounting substrate 23, a charging circuit 24, and a connection member 25.

The housing 11 is a case that houses components. The housing 11 houses, for example, the display unit 12, the operation unit 13, the pump 14, the on-off valve 16, the pressure sensor 17, the battery 18, the communication unit 19, the memory 20, the processor 21, the flow path plate unit 22, and the mounting substrate 23.

The housing 11 includes, for example, an exterior case 31, a windshield 32 covering an upper opening of the exterior case 31, and a back cover 35 covering a lower side of the exterior case 31.

The exterior case 31 is formed in, for example, a cylindrical shape, a rectangular cylindrical shape, or a polygonal cylindrical shape. In the present embodiment, an example is explained in which the exterior case 31 is formed in the cylindrical shape. The exterior case 31 includes a pair of lugs 31a provided at symmetrical positions in the circumferential direction of the outer peripheral surface and a spring bar 31b provided between the two pair of lugs 31a. The windshield 32 is a circular glass plate.

The display unit 12 is disposed immediately under the windshield 32. The display unit 12 is electrically connected to the processor 21. The display unit 12 is, for example, a liquid crystal display or an organic electroluminescence display. The display unit 12 displays various types of information including a date and time and measurement results of blood pressure values such as maximum blood pressure and minimum blood pressure, a heart rate, and the like.

The operation unit 13 is configured to be capable of inputting an instruction from a user. The operation unit 13 includes, for example, a plurality of buttons 41 provided on the housing 11, a sensor that detects operations on the buttons 41, and a touch panel 43 provided on the display unit 12 or the windshield 32. By being operated by the user, the operation unit 13 converts an instruction to an electric signal. The sensor and the touch panel 43 are electrically connected to the processor 21 and outputs electric signals to the processor 21.

The pump 14 is, for example, a piezoelectric pump. The pump 14, for example, compresses air and supplies the compressed air to the cuff structure 7 via the flow path plate unit 22. The pump 14 is electrically connected to the processor 21. The pump 14 is disposed to overlap the flow path plate unit 22. For example, the pump 14 is attached to the main surface of the flow path plate unit 22.

The acceleration sensor 15 is, for example, a three-axis acceleration sensor. The acceleration sensor 15 measures, for example, acceleration and outputs an analog signal. The acceleration sensor 15 is connected to the processor 21 via, for example, an A/D conversion circuit.

The on-off valve 16 is, for example, a valve for safety that releases air supplied to a pressing cuff 71 and a sensing cuff 73 explained below of the cuff structure 7 to the atmosphere. The on-off valve 16 is connected to, for example, a branch flow path 22c1 of a first flow path 22c explained below, which connects the pump 14 and on-off valve 16 to the pressing cuff 71, of the flow path plate unit 22. The on-off valve 16 is electrically connected to the processor 21. For example, the on-off valve 16 is opened and closed by the control of the processor 21.

The on-off valve 16 is, for example, a quick exhaust valve capable of performing quick exhaust, the opening degree of the on-off valve 16 or the opening area of the first flow path 22c being set such that a fluid resistance becomes as low as possible. When air is supplied to the pressing cuff 71 and the sensing cuff 73 at the time of measuring blood pressure, the on-off valve 16 is switched to a closed state by being controlled by the processor 21. When air is exhausted from the pressing cuff 71 and the sensing cuff 73, the on-off valve 16 is switched from the closed state to an open state by being controlled by the processor 21 The on-off valve 16 may be formed to be capable of adjusting the opening degree. Note that the on-off valve 16 may be integrally provided on the inside of the housing of the pump 14.

The pressure sensor 17 is fluidly connected to a flow path unit 22a. The pressure sensor 17 detects, for example, a pressure of the sensing cuff 73 of the cuff structure 7 via the flow path unit 22a. The pressure sensor 17 is electrically connected to the processor 21 via, for example, an A/D conversion circuit, converts the detected pressure into an electric signal, and outputs the electric signal to the processor 21.

The battery 18 is, for example, a secondary battery such as a lithium-ion battery. The battery 18 is electrically connected to the processor 21. The battery 18 supplies electric power to the processor 21. The battery 18 supplies driving power to the structural components of the processor 21 and to the display unit 12, the operation unit 13, the pump 14, the acceleration sensor 15, the on-off valve 16, the pressure sensor 17, and the communication unit 19 via the processor 21.

The communication unit 19 is configured to be capable of transmitting and receiving information to and from an external device wirelessly or by wire. The communication unit 19 is, for example, a wireless communication module conforming to the wireless communication standard. The communication unit 19 transmits information such as information controlled by the processor 21 and measured blood pressure values and pulse to an external device and receives a program or the like for software update from the external device and transmits the program and the like to the control unit. In the present embodiment, the external device is, for example, an external terminal such as a smartphone, a tablet terminal, a personal computer, and a smart watch.

In the present embodiment, the communication unit 19 and the external device may be directly connected or may be connected via a network. The communication unit 19 and the external device may be connected via a mobile communication network such as 4G or 5G and a wireless communication line such as Wimax or Wi-Fi (registered trademark). The communication unit 19 and the external device may be connected by wireless communication means such as Bluetooth (registered trademark), NFC (Near Filed Communication), or infrared communication. Further, the communication unit 19 and the external device may be connected via a wired communication line such as a USB (Universal Serial Bus) or a LAN (Local Area Network) connection by a cable. Therefore, the communication unit 19 may be configured to include a plurality of communication means such as a wireless antenna and a micro-USB connector.

The memory 20 includes, for example, a RAM (Random Access Memory) and a ROM (Read Only Memory). The memory 20 stores various types of data. For example, the memory 20 pre-stores, for example, program data for controlling the entire blood pressure measurement device 1 and the pump 14, settings data for setting various functions of the blood pressure measurement device 1, and calculation data for calculating a blood pressure value and a pulse from the pressure measured by the pressure sensor 17 to be changeable.

The processor 21 controls the operation of the entire blood pressure measurement device 1 and the operations of the pump 14 and the on-off valve 16 based on programs stored in the memory 20 and causes the blood pressure measurement device 1, the pump 14, and the on-off valve 16 to execute predetermined operations (functions).

The processor 21 executes a predetermined arithmetic operation, an analysis, processing, and the like according to read programs. The processor 21 is an arithmetic device such as a CPU.

The flow path plate unit 22 is accommodated in the housing 11. The flow path plate unit 22 fluidly connects the pump 14, the on-off valve 16, and the pressure sensor 17 and the cuffs 71 and 73 explained below of the cuff structure 7. The flow path plate unit 22 includes the flow path unit 22a on the inside thereof. The flow path unit 22a fluidly connects the cuffs 71 and 73 and the atmosphere via the on-off valve 16.

The flow path plate unit 22 includes a first flow path plate 131, a second flow path plate 132, and an attachment member 133 that attaches the first flow path plate 131 and the second flow path plate 132. The flow path unit 22a is configured by the first flow path plate 131, the second flow path plate 132 and attachment member 133.

The first flow path plate 131 has one surface formed in a planar shape, the one surface being opposed to the second flow path plate 132. On a surface of the first flow path plate 131, which is opposite to the surface thereof opposed to the second flow path plate 132, for example, the pump 14, the on-off valve 16, and the pressure sensor 17 are fixed. In the first flow path plate 131, for example, a hole fluidly connected to the pump 14, a hole fluidly connected to the on-off valve 16, and a hole fluidly connected to the pressure sensor 17 are formed. The first flow path plate 131 is formed of a conductive material having conductivity, for example, a metallic material. The first flow path plate 131 is, for example, a metallic plate. The thickness of the first flow path plate 131 is, for example, 0.4 mm. The pump 14 is attached to a surface of first flow path plate 131 opposite to a surface facing the second flow path plate 132.

The second flow path plate 132 has one surface formed in a planar shape, the one surface being opposed to the first flow path plate 131. The opposed surfaces of the second flow path plate 132 and first flow path plate 131 have substantially identical outer shapes. The second flow path plate 132 is formed of a conductive material having conductivity, for example, a metallic material. The thickness of the second flow path plate 132 is, for example, 0.4 mm.

The attachment member 133 attaches the first flow path plate 131 and the second flow path plate 132.

The attachment member 133 includes a notch 133a that forms the flow path unit 22a in conjunction with the first flow path plate 131 and the second flow path plate 132 when the first flow path plate 131 and the second flow path plate 132 are attached. Specifically, the attachment member 133 is formed to have substantially the same shape as the outer shape of each of the opposed surfaces of the flow path plates 131 and 132 and is formed by providing the notch 133a that partly opens in a shape corresponding to the flow path unit 22a.

The attachment member 133 is, for example, a double-sided tape. A material with air-tightness is used for the attachment member 133. For example, the attachment member 133 is a double-sided tape including a base member formed of a material with air-tightness such as an acrylic foam material. The thickness of the attachment member 133 is, for example, 0.2 mm.

The attachment member 133 is aligned with the first flow path plate 131 and the second flow path plate 132, for example, by using alignment holes provided in the first flow path plate 131 and second flow path plate 132. The attachment member 133 is fixed to the first flow path plate 131 and second flow path plate 132, for example, by manual work by a worker.

As a specific example, the flow path plate unit 22 is connected to the pump 14 and the cuff structure 7.

The flow path unit 22a of the flow path plate unit 22 includes, for example, a first flow path 22c and a second flow path 22d.

The first flow path 22c fluidly connects the pump 14 and the on-off valve 16 to the pressing cuff 71. In a concrete example, the first flow path 22c includes a branch flow path 22c1 that is branched on the secondary side of the pump 14. The branch flow path 22c1 is connected to the on-off valve 16. The second flow path 22d is a flow path connected to the pressure sensor 17.

In a concrete example, a first hole 131a, a second hole 131b, and a third hole 131c are formed in the first flow path plate 131. The holes 131a, 131b and 131c penetrate the first flow path plate 131.

The first hole 131a communicates with a discharge port of the pump 14. The first hole 131a constitutes a portion of the first flow path 22c. The first hole 131a is disposed, for example, on a central side of the first flow path plate 131. The second hole 131b is connected to the pressure sensor 17. The second hole 131b constitutes a portion of the second flow path 22d. The second hole 131b is disposed, for example, on an outer edge side of the first flow path plate 131. The third hole 131c is connected to the on-off valve 16. The third hole 131c constitutes a portion of the branch flow path 22c1. The third hole 131c is disposed, for example, on an outer edge side of the first flow path plate 131.

The second flow path plate 132 includes, for example, a flow path plate body 132a and a nozzle 132b. The flow path plate body 132a has one surface formed in a planar shape, the one surface being opposed to the first flow path plate 131. The opposed surfaces of the flow path plate body 132a and the first flow path plate 131 have substantially identical outer shapes. The flow path plate body 132a is, for example, a metallic plate.

The nozzle 132b is provided on a surface of the flow path plate body 132a on the opposite side to the attachment member 133. The nozzle 132b is connected to the cuff structure 7. The nozzle 132b is formed of, for example, a resin. The nozzle 132b is formed as one piece with the flow path plate body 132a, for example, by insert molding.

In a concrete example, the nozzle 132b includes a first nozzle 132b1 and a second nozzle 132b2. The first nozzle 132b1 communicates with the first flow path 22c. The first nozzle 132b1 is connected to the pressing cuff 71. The second nozzle 132b2 communicates with the second flow path 22d. The second nozzle 132b2 is connected to the sensing cuff 73.

The mounting substrate 23 is accommodated in the housing 11. Various electronic components are mounted on the mounting substrate 23. The mounting substrate 23 includes, for example, a printed circuit board 231. Digital circuit components such as the communication unit 19, the memory 20, and the processor 21 and analog circuit components such as the acceleration sensor 15, the pressure sensor 17, a PPG sensor, and an electrocardiographic sensor are mounted on the mounting substrate 23. The mounting substrate 23 is a PCB (Printed Circuit Board) on which a plurality of electronic components are mounted. The mounting substrate 23 supplies fluid to the cuff structure 7 with the mounted various electronic components.

The printed circuit board 231 is, for example, a rigid substrate. For example, electronic components are mounted on both surfaces of the printed circuit board 231. Patterned wires are formed on the printed circuit board 231. The shape in the main surface direction of the printed circuit board 231 is larger than, for example, the main surface of the pump 14. The printed circuit board 231 is, for example, a multilayer substrate and includes a ground layer. GND is provided on the printed circuit board 231. The printed circuit board 231 is fixed to the flow path plate unit 22 via, for example, a spacer 232.

The charging circuit 24 includes, for example, an antenna unit 241, a power receiving unit 242, and a charging unit 243. The charging circuit 24 charges the battery 18 with wireless power supply. For example, the charging circuit 24 receives transmission power transmitted from an antenna unit of a power transmission apparatus provided outside and charges the battery 18.

The antenna unit 241 receives transmission power from the antenna unit of the power transmission apparatus. The power receiving unit 242 rectifies the power received by the antenna unit 241 and supplies the rectified power to the charging unit 243. The charging unit 243 supplies the power supplied from the power receiving unit 242 to the battery 18 as power for charging. For example, the charging unit 243 converts the power supplied from the power receiving unit 242 into power having a predetermined current value and a predetermined voltage value and supplies the converted power to the battery 18. The power receiving unit 242 and/or the charging unit 243 converts the power received by the antenna unit 241 from AC into DC.

The connection member 25 connects the flow path plate unit 22 and the GND of the printed circuit board 231. The connection member 25 is formed of, for example, a metallic material. The shape of the connection member 25 is set according to the shapes of and a positional relation between the flow path plate unit 22 and the printed circuit board 231. Various methods such as soldering, screwing, adhesion using an adhesive including a conductive material, sandwiching, insertion, and mechanical joining such as fitting can be applied as a method of connecting the connection member 25 to the flow path plate unit 22 and the printed circuit board 231. The connection member 25 is connected to at least one of the first flow path plate 131 and the second flow path plate 132. In the example illustrated in FIG. 3, the connection member 25 is fixed to, for example, the first flow path plate 131 of the flow path plate unit 22 by a screw 25a and is fixed to the land of the printed circuit board 231 by soldering.

As illustrated in FIG. 1, the belt 4 includes a first belt 61 provided on one pair of lugs 31a and a spring bar 31b and a second belt 62 provided on the other pair of lugs 31a and a spring bar 31b.

The first belt 61 is referred to as a so-called a parent and is configured like a band. The first belt 61 includes a buckle 61c provided at one end portion thereof. The first belt 61 is rotatably held on the exterior case 31. The buckle 61c includes a frame-shaped body 61d having a rectangular frame shape and a buckle tongue 61e rotatably attached to the frame-shaped body 61d. The second belt 62 is referred to as a so-called tip of a blade and is formed like a band having such a width as to be insertable into the frame-shaped body 61d. The second belt 62 includes a plurality of small holes 62a into which the buckle tongue 61e is insertable.

In the belt 4, the second belt 62 is inserted into the frame-shaped body 61d and the buckle tongue 61e is inserted into the small hole 62a, whereby the first belt 61 and the second belt 62 are connected as one piece and form, together with the exterior case 31, an annular shape along the circumferential direction of the wrist.

The curler 5 is formed of a resin material and is configured in a band-like shape that curves in such a manner as to follow along the circumferential direction of the wrist. For example, one end of the curler 5 is fixed to the wrist side of the device main body 3.

The curler 5 has hardness appropriate to provide flexibility and shape retainability. Here, the flexibility refers to deformation of the shape of the curler 5 in a radial direction at the time of application of an external force of the belt 4 to the curler 5. The shape retainability refers to the ability of the curler 5 to maintain a pre-imparted shape when no external force is applied to the curler 5. The cuff structure 7 is disposed on the inner circumferential surface of the curler 5.

As illustrated in FIG. 1, the cuff structure 7 includes, for example, a pressing cuff 71, a back plate 72, and a sensing cuff 73. In the cuff structure 7, the pressing cuff 71, the back plate 72 and the sensing cuff 73 are stacked and formed as one piece. The cuff structure 7 is fixed to the inner surface of the curler 5.

In the example of the present embodiment, in the cuff structure 7, the pressing cuff 71 is connected to the sensing cuff 73 via the fluid control unit 9 and the sensing cuff 73 is connected to the atmosphere via the fluid control unit 9.

The pressing cuff 71 is connected to the flow path plate unit 22. The pressing cuff 71 is fluidly connected to the pump 14 via the flow path plate unit 22. One major surface of the pressing cuff 71 is fixed to the inner surface of the curler 5. For example, the pressing cuff 71 is attached to the inner surface of the curler 5 by a double-sided tape or an adhesive. By inflating, the pressing cuff 71 presses the back plate 72 and the sensing cuff 73 against the living body side.

The pressing cuff 71 includes, for example, an air bag 81.

The air bag 81 is a bag-like structure. In the present embodiment, since the blood pressure measurement device 1 is configured to use air with the pump 14, a description is given of a case of using the air bag. However, in a case of using fluid other than air, the bag-like structure may be a fluid bag such as a liquid bag.

The back plate 72 is stuck to a wrist-side surface of the pressing cuff 71 by a double-sided tape, an adhesive, or the like. The back plate 72 is formed of a resin material and is formed in a plate shape. The back plate 72 is formed of, for example, polypropylene and is formed in a plate shape with thickness of approximately 1 mm. The back plate 72 has a shape follow-up characteristic.

Here, the shape follow-up characteristic means a function by which the back plate 72 is deformable in such a manner as to imitate the shape of a contacted part of the wrist to be placed. The contacted part of the wrist means an area of contact with the back plate 72. The contact in this context means both direct contact and indirect contact.

The sensing cuff 73 is fixed to a wrist-side main surface of the back plate 72. The sensing cuff 73 comes in direct contact with an area where the artery of the wrist is present. The sensing cuff 73 is formed to have an identical shape to the back plate 72 or to have a smaller shape than the back plate 72 in the longitudinal direction and the width direction of the back plate 72. By inflating, the sensing cuff 73 presses an area where a palm-side artery of the wrist is present. By the inflated pressing cuff 71, the sensing cuff 73 is pressed against the living body side via the back plate 72.

In a concrete example, the sensing cuff 73 includes one air bag 91 and a flow path body 92.

Here, the air bag 91 is a bag-like structure. In the present embodiment, since the blood pressure measurement device 1 is configured to use air with the pump 14, a description is given of a case of using the air bag. However, in a case of using fluid other than air, the bag-like structure may be a liquid bag or the like.

The air bag 91 is constituted in a rectangular shape that is long in one direction. The air bag 91 is formed by, for example, combining two sheet members long in one direction and thermally welding edge portions thereof.

The flow path body 92 is, for example, integrally provided at a portion of one edge portion of the air bag 91 in the longitudinal direction. The flow path body 92 is provided at the end portion of the air bag 91 near the device main body 3. The flow path body 92 is formed in a shape that is long in one direction with width smaller than the dimension in the width direction of the air bag 91. The flow path body 92 includes, for example, a connection portion at a distal end thereof. The flow path body 92 is connected to the flow path unit 22a via the connection portion and forms a flow path between the flow path unit 22a and the air bag 91.

The fluid control unit 9 controls, for example, a supply amount of air supplied to the cuffs 71 and 73. The fluid control unit 9 is, for example, a fluid resistor such as an orifice or a check valve. In the example of the present embodiment, the fluid control unit 9 includes, for example, a plurality of flow rate resistors. The fluid control unit 9 controls a pressure ratio of air between the two cuffs 71 and 73 to be constant according to a flow rate resistance ratio between a plurality of flow rate resistors.

The fluid control unit 9 causes a pressure difference between the pressing cuff 71 and the sensing cuff 73 with the plurality of flow path resistors and controls a pressure ratio between the pressing cuff 71 and the sensing cuff 73 to be constant. The fluid control unit 9 sets a flow rate resistance ratio according to characteristics of the cuffs 71 and 73 of the blood pressure measurement device 1.

With the blood pressure measurement device 1 configured as explained above, the flow path plate unit 22 can be used as the GND by connecting the GND of the printed circuit board 231 and the flow path plate unit 22 formed of the metallic material using the connection member 25. The blood pressure measurement device 1 is capable of taking measures against static electricity and noise using the flow path plate unit 22. Since the flow path plate unit 22 is a component necessary for exerting the function of the blood pressure measurement device 1, the blood pressure measurement device 1 does not need to be provided with another component for measures against static electricity besides the connection member 25 by taking measures against static electricity using the flow path plate unit 22. Therefore, the blood pressure measurement device 1 can prevent an increase in the size of the device main body 3 and an increase in manufacturing cost.

The connection member 25 is configured to be connected to at least one of the first flow path plate 131 and the second flow path plate 132 formed of the metallic material. Therefore, the connection member 25 only has to be connected to a part of either the first flow path plate 131 or the second flow path plate 132. Flexibility in designing a connection position and the like is high.

Since it is unnecessary to use the housing 11 for measures against static electricity, flexibility of the housing 11 is also improved by forming the housing 11 using a resin material or using the housing 11 as an electrode. Note that, when the housing 11 is formed of a conductive material and is not used as an electrode, the housing 11 can also be used as measures against static electricity in addition to the flow path plate unit 22.

As explained above, with the blood pressure measurement device 1 according to the present embodiment, by connecting the flow path plate unit 22 to the GND of the mounting substrate 23 using the connection member 25, it is possible to suppress an increase in size and cost and take measures against static electricity.

Note that the present invention is not limited to the example of the embodiment explained above. For example, in the example explained above, an example is explained in which the connection member 25 is connected to the first flow path plate 131 of the flow path plate unit 22. However, the present invention is not limited thereto. For example, the connection member 25 may be connected to the second flow path plate 132 of the flow path plate unit 22.

The connection member 25 may be configured to be connected to both the first flow path plate 131 and the second flow path plate 132. For example, the attachment member 133 may be formed of a material containing a conductive material. The connection member 25 may be connected to one of the first flow path plate 131 and the second flow path plate 132. With the configuration in which both the first flow path plate 131 and the second flow path plate 132 are connected to the connection member 25, since the volume of the flow path plate unit 22 connected to the GND of the mounting substrate 23 (the printed circuit board 231) increases, it is possible to suitably take measures against static electricity.

A plurality of connection members 25 may be provided. For example, the pin 26 illustrated in FIG. 3 used for the printed circuit board 231 may be configured to connect the GND of the printed circuit board 231 and the flow path plate unit 22 in addition to the connection member 25 or in place of the connection member 25.

In the example explained above, the configuration in which the connection member 25 connects the flow path plate unit 22 and the GND of the printed circuit board 231 is explained. However, the present invention is not limited thereto. The detailed configuration of the connection member 25 can be set as appropriate if the connection member 25 can directly or indirectly connect the flow path plate unit 22 and the GND of the printed circuit board 231. That is, the connection member 25 may be configured to connect the flow path plate unit 22 and GND of another component connected to the GND of the printed circuit board 231.

Note that the present invention is not limited to the embodiment explained above and can be variously modified in an implementation stage without departing from the gist of the present invention. Embodiments may be implemented by being combined as appropriate as much as possible. In that case, a combined effect can be obtained. Further, the embodiment explained above includes inventions at various stages. Various inventions can be extracted by an appropriate combination of a disclosed plurality of constituent elements. For example, even if some constituent elements are deleted from all the constituent elements explained in the embodiment, if the problems can be solved and the effects can be obtained, a configuration from which the constituent elements are deleted can be extracted as an invention.

### REFERENCE SIGNS LIST

1 BLOOD PRESSURE MEASUREMENT DEVICE

3 DEVICE MAIN BODY

4 BELT

5 CURLER

7 CUFF STRUCTURE

9 FLUID CONTROL UNIT

11 HOUSING

12 DISPLAY UNIT

13 OPERATION UNIT

14 PUMP

15 ACCELERATION SENSOR

16 ON-OFF VALVE

17 PRESSURE SENSOR

18 BATTERY

19 COMMUNICATION UNIT

20 MEMORY

21 PROCESSOR

22 FLOW PATH PLATE UNIT

22a FLOW PATH UNIT

22c FIRST FLOW PATH

22c1 BRANCH FLOW PATH

22d SECOND FLOW PATH

23 MOUNTING SUBSTRATE

24 CHARGING CIRCUIT

25 CONNECTION MEMBER

25a SCREW

26 PIN

31 EXTERIOR CASE

31a LUG

31b SPRING BAR

32 WINDSHIELD

35 BACK COVER

41 BUTTON

43 TOUCH PANEL

61 FIRST BELT

61c BUCKLE

61d FRAME-SHAPED BODY

61e BUCKLE TONGUE

62 SECOND BELT

62a SMALL HOLE

71 PRESSING CUFF

72 BACK PLATE

73 SENSING CUFF

81 AIR BAG

91 AIR BAG

92 FLOW PATH BODY

131 FIRST FLOW PATH PLATE

131a FIRST HOLE

131b SECOND HOLE

131c THIRD HOLE

132 SECOND FLOW PATH PLATE

132a FLOW PATH PLATE BODY

132b NOZZLE

132b1 FIRST NOZZLE

132b2 SECOND NOZZLE

133 ATTACHMENT MEMBER

133a NOTCH

231 PRINTED CIRCUIT BOARD

232 SPACER

241 ANTENNA UNIT

242 POWER RECEIVING UNIT

243 CHARGING UNIT

What is claimed is:

1. A blood pressure measurement device comprising:
a housing;
a flow path plate unit accommodated in the housing and at least partially formed of a conductive material;
a pump connected to the flow path plate unit;
a pressure sensor connected to the flow path plate unit;
a cuff connected to the flow path plate unit and fluidly connected to the pump and the pressure sensor via the flow path plate unit;
a printed circuit board accommodated in the housing and including GND; and
at least one connection member that connects the GND of the printed circuit board and a part of the flow path plate unit formed of the conductive material.

2. The blood pressure measurement device according to claim 1, wherein
the flow path plate unit includes:
a first flow path plate formed of a metallic material;
a second flow path plate formed of a metallic material; and
an attachment member that attaches the first flow path plate and the second flow path plate, and
the connection member is connected to at least one of the first flow path plate and the second flow path plate.

3. The blood pressure measurement device according to claim 2, wherein the attachment member includes a conductive material.
